(19) United States
(12) Patent Application Publication
(10) Pub. No.: US 2006/0264491 A1
(43) Pub. Date: Nov. 23, 2006

(54) Telmisartan production process

(75) Inventors: Michael Brand, Ra'anana (IL); Ada Salman, Ramat Gan (IL); Yael Gafni, Rosh Ha'ayin (IL); Michal Noiman, Nes Ziona (IL); Alex Weisman, Kiryat Ekron (IL); Joseph Kaspi, Givatayim (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: CHEMAGIS LTD., Bnei Brak (IL)

(21) Appl. No.: 11/449,087

(22) Filed: Jun. 8, 2006

(51) Int. Cl. A61K 31/4184; C07D 403/02

(52) U.S. Cl. 514/394; 548/305.4

(57) ABSTRACT

The present invention provides an intermediate and a process for preparing Telmisartan, which overcomes the drawbacks of conventional methods and produces Telmisartan in high purity and yield.

### BACKGROUND OF THE INVENTION

Telmisartan, 4′-[2-n-propyl-4-methyl-6-(1-methylbenzimidazol-2-yl)benzimidazol-1-ylmethyl]biphenyl-2-carboxylic acid, is a non-peptide ATI-subtype angiotensin II receptor antagonist having the structural formula I (below).

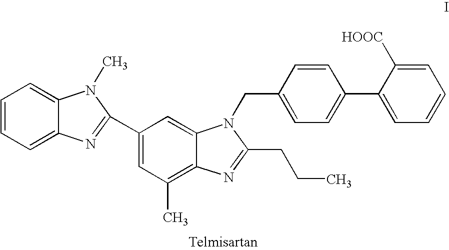

Telmisartan is indicated for the treatment of hypertension, either alone or in combination with diuretic agents. It is effective in once-daily dosing, so that significant blood-pressure lowering effect is observed even during the last 6 hours of the dosing interval. Telmisartan is marketed in the US by Boehringer Ingelheim under the names MICARDIS® and MICARDIS-HCT®.

The synthesis of Telmisartan was first described in U.S. Pat. No. 5,591,762 (hereinafter the '762 patent) by hydrolysis of the tertiary-butyl ester precursor of Telmisartan, in particular, tert-butyl 4′-[2-n-propyl-4-methyl-6-(1-methylbenzimidazol-2-yl)benzimidazol-1-ylmethyl]biphenyl-2-carboxylic acid using trifluoroacetic acid in N,N-dimethylformamide (DMF).

Chinese patent application CN 1344712 (hereinafter the '712 application) describes the preparation of Telmisartan by reacting 2-n-propyl-4-methyl-6-(1-methylbenzimidazol-2-yl)benzimidazole (compound II) with 4′-(bromomethyl)-[1,1′-biphenyl]-2-carboxylic acid methyl or ethyl ester (compound III), via nucleophilic substitution, to give the carboxylic ester derivatives of Telmisartan (compound IV), followed by hydrolysis, to afford Telmisartan.

The process for preparing Telmisartan according to the '712 application is depicted in Scheme 1.

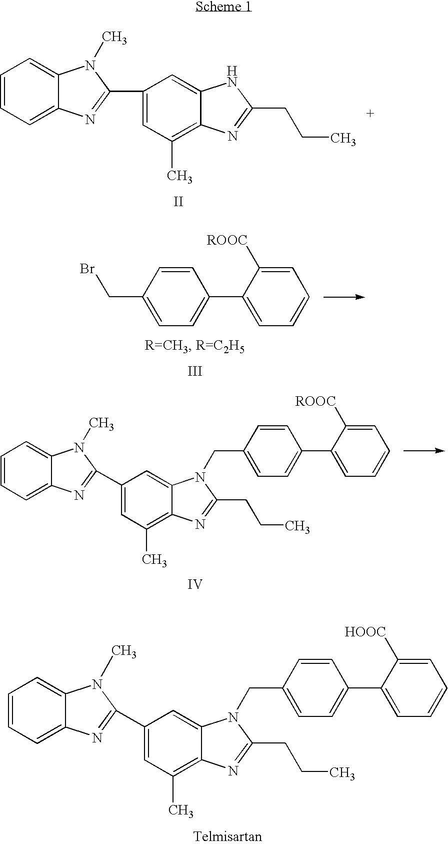

WO 2006/044648 describes synthetic route, which is similar to the synthesis described in the '712 application, using a low boiling solvent and a PTC.

Chinese patent application CN 1412183 (hereinafter the '183 application) describes a process for preparing Telmisartan, which includes reacting compound II with 4′-(bromomethyl)-[1,1′-biphenyl]-2-carbonitrile (compound V), to afford the carbonitrile derivative of Telmisartan, i.e., 4′-[(1,4′-dimethyl-2′-propyl[2,6′-bi-1H-benzimidazol]-1′-yl)methyl]-[1,1′-biphenyl]-2-carbonitrile (compound VI), followed by hydrolysis of the cyano group of compound VI, to afford Telmisartan.

The process for preparing Telmisartan according to the '183 application is depicted in Scheme 2.

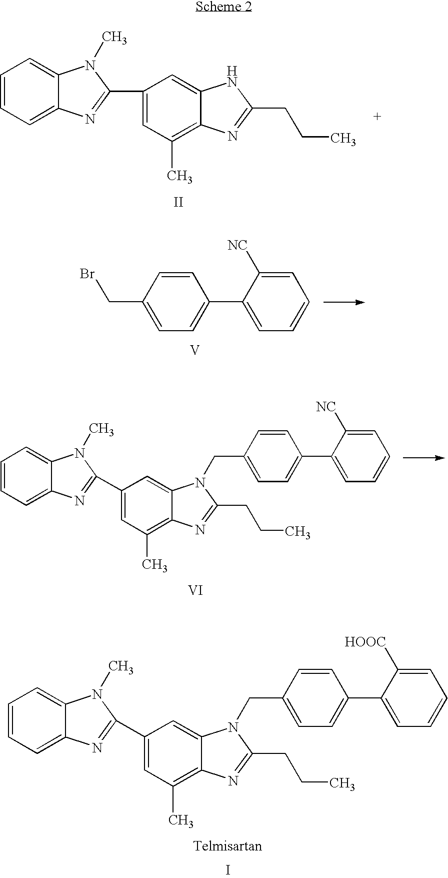

Example 3 of the '183 application teaches producing Telmisartan by hydrolyzing compound VI using a mixture of glacial acetic acid (e.g., 150 ml) with a two-fold volumetric excess of concentrated hydrochloric acid (e.g., 300 ml) per 0.05 moles of compound IV at a temperature of 100° C., which is not an environmentally friendly process. Furthermore, upon crystallization of the crude Telmisartan from N,N-dimethylformamide (DMF), a material having a purity of 99.0% was obtained, which is insufficient for pharmaceutical purposes.

US 2004/0236113 (hereinafter the '113 application) also describes a process for preparing Telmisartan, which includes reacting compound II with 4′-(bromomethyl)-[1,1′-biphenyl]-2-carbonitrile (compound V) to afford the carbonitrile derivative of Telmisartan, i.e., 4′-[(1,4′-dimethyl-2′-propyl [2,6′-bi-1H-benzimidazol]-1′-yl)methyl]-[1,1′-biphenyl]-2-carbonitrile (compound VI). The cyano group of compound VI is hydrolyzed using a mixture of potassium hydroxide and aqueous ethylene glycol at 160° C., followed by distilling off the solvent and adding concentrated hydrochloric acid, to obtain Telmisartan hydrochloride, which crystallizes upon cooling. The Telmisartan hydrochloride is separated and further dissolved in acetic acid under reflux, hot filtered through charcoal, and washed with acetic acid. Then, 4N NaOH is added dropwise at 80° C.-90° C., and the solution is diluted and cooled, after which the free Telmisartan is filtered off, washed and dried. The purity of the free Telmisartan is not disclosed in the example of the '113 application.

There is a need in the art for a process for preparing Telmisartan and an intermediate therefor, which on the one hand avoids the need to prepare the hydrochloride salt (which is a lengthy process) and, on the other hand, utilizes an environmentally friendly process that yields highly pure Telmisartan in high yield. The present invention provides such an intermediate and process.

### BRIEF SUMMARY OF THE INVENTION

The present invention provides a process for preparing highly pure Telmisartan in high yield, which process overcomes the limitations of conventional methods for preparing Telmisartan, and yet is straight-forward and environmentally friendly.

In one embodiment, the process of the present invention includes converting 4′-[(1,4′-dimethyl-2′-propyl [2,6′-bi-1H-benzimidazol]-1′-yl)methyl]-[1,1′-biphenyl]-2-carboxamide (compound VII) into Telmisartan (e.g., via hydrolysis of the carboxamide); isolating the crude Telmisartan; and, optionally, purifying the crude Telmisartan (e.g., via crystallization). Preferably, compound VII is prepared by a process, which includes reacting 4′-[(1,4′-dimethyl-2′-propyl [2,6′-bi-1H-benzimidazol]-1′-yl)methyl]-[1,1′-biphenyl]-2-carbonitrile (compound VI) with an acid or a base in at least one solvent; isolating compound VII (e.g., by separating the product as a solid, e.g., via filtration; washing the product with at least one solvent; and, optionally, drying.

An exemplary process of the present invention includes reacting compound VII with a base in an organic solvent; distilling off the majority of the solvent and adding an anti-solvent; treating the reaction mixture with charcoal and Celite and filtering; adding aqueous acetic acid to obtain a suspension; cooling the suspension and separating the solid product by filtration; washing the product with at least one solvent, and drying.

### DETAILED DESCRIPTION OF THE INVENTION

The inventors of the present invention have discovered a novel process for preparing highly pure Telmisartan in high yield, which overcomes the limitations of conventional methods for preparing Telmisartan. The process of the present invention preferably includes converting 4′-[(1,4′-dimethyl-2′-propyl[2,6′-bi-1H-benzimidazol]-1′-yl)methyl]-[1,1′- biphenyl]-2-carboxamide (compound VII) into Telmisartan. In one embodiment, the process of the present invention includes hydrolyzing compound VII, preferably using a mixture of a base and propylene glycol, which is an environmentally friendly ICH class 3 solvent.

In one embodiment, the process of present invention includes converting 4′-[(1,4′-dimethyl-2′-propyl [2,6′-bi-1H-benzimidazol]-1′-yl)methyl]-[1,1′- biphenyl]-2-carboxamide (compound VII) into Telmisartan; isolating the product as crude Telmisartan; and, optionally, purifying the Telmisartan (preferably by crystallizing the Telmisartan).

Scheme 3 depicts an exemplary process for preparing Telmisartan in accordance with the present invention.

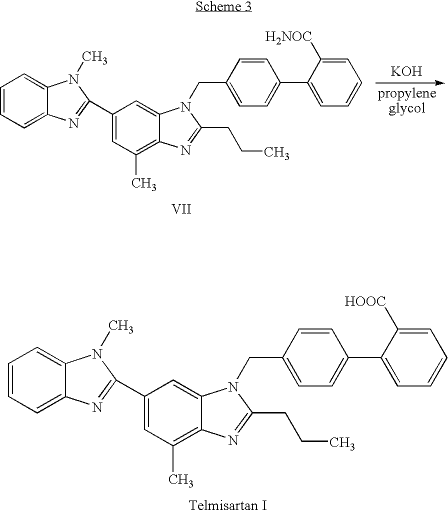

Compound VII can be prepared by any suitable process, including processes that utilize reagents and synthetic transformations, which are well known to those of ordinary skill in the art. In one embodiment, compound VII is prepared by the process depicted in Scheme 4, wherein the starting material 4′-(bromomethyl)-[1,1′-biphenyl]-2-carbonamide (compound VIII) can be easily obtained from 4′-methyl-[1,1′-bipheyl]-2-carbonitrile (compound IX), which is commercially available.

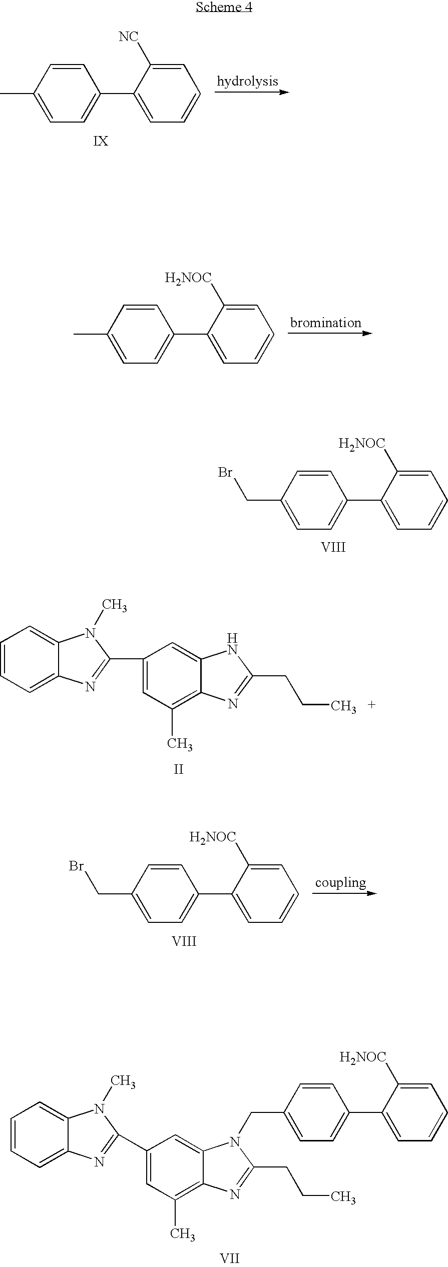

The process depicted in Scheme 4 is advantageous in that the amides are crystalline materials and can be easily obtained in high purity, e.g., by crystallization.

An alternative process for producing compound VII includes reacting 4′-[(1,4′-dimethyl-2′-propyl [2,6′-bi-1H-benzimidazol]-1′-yl)methyl]-[1,1′-biphenyl]-2-carbonitrile (compound VI) with an acid or a base in a at least one solvent; separating the solid product by filtration; washing with at least one solvent and drying.

Suitable bases, which can be used for converting compound VI into compound VII include, for example, sodium hydroxide, potassium hydroxide, lithium hydroxide, barium hydroxide, cesium hydroxide, sodium bicarbonate, potassium bicarbonate, sodium carbonate, potassium carbonate, cesium carbonate, and the like, and combinations thereof. A preferred base for converting compound VI into compound VII is sodium hydroxide.

Suitable acids, which can be used for converting compound VI into compound VII, include, for example, mineral and organic acids, e.g., hydrochloric acid, hydrobromic acid, sulfuric acid, acetic acid, methanesulfonic acid, trifluoroacetic acid, and the like, and combinations thereof.

Suitable solvents, which can be used for washing the solid product (e.g., compound VII obtained in accordance with the procedures described herein) include, e.g., methanol, ethanol, 1-propanol, 2-propanol, water and mixtures thereof. Preferably, compound VII is washed with a 2:1 (v/v) mixture of ethanol/water.

The crude compound VII is preferably obtained in a yield of at least about 90% and a purity of at least 98.9%.

An exemplary process of the present invention includes reacting compound VII with a base in an organic solvent; distilling off the majority (e.g., at least a substantial portion) of the solvent and adding an anti solvent; treating the reaction mixture with charcoal and Celite and filtering; adding aqueous acetic acid to obtain a suspension; cooling the suspension and obtaining the solid product by filtration; washing with at least one solvent; and drying, to produce Telmisartan.

Suitable bases, which can be used for converting compound VII into Telmisartan, include, for example, sodium hydroxide, potassium hydroxide, lithium hydroxide, barium hydroxide, cesium hydroxide, and combinations thereof. A preferred base for converting compound VII into Telmisartan is potassium hydroxide.

Solvents suitable for use in the reaction for converting compound VII into Telmisartan include, for example, methanol, ethanol, 1-propanol, 2-propanol, water, THF, N,N-dimethylacetamide (DMA), ethylene glycol, propylene glycol, and the like, and mixtures thereof. Preferably, propylene glycol is used as a solvent in the reaction for converting compound VII into Telmisartan.

Suitable solvents, which can be used for washing Telmisartan obtained in accordance with the process of the present invention include, for example, methanol, ethanol, 1-propanol, 2-propanol, water, and the like, and mixtures thereof. A preferred solvent for washing Telmisartan obtained in accordance with the present invention, is water. In addition, a preferred anti-solvent, which can be used in accordance with the process of the present invention, is water.

The process of the present invention preferably produces a crude Telmisartan product in a yield of at least about 84% and a purity of at least about 99.3%. Further, the process of the present invention preferably produces Telmisartan containing less than about 0.25% (wt %) residual ammonia, and more preferably containing less than 0.1% (wt %) residual ammonia, and most preferably containing less than 0.02% (wt %) residual ammonia.

The following examples further illustrate the invention but, of course, should not be construed as in any way limiting its scope.

### EXAMPLE 1

This example illustrates a method for preparing 4′-[(1,4′-dimethyl-2′-propyl [2,6′-bi-1H-benzimidazol]-1′-yl)methyl]-[1,1′- biphenyl]-2-carboxamide (compound VII).

A two-necked reaction vessel equipped with a reflux condenser and a thermometer was charged with 4′-[(1,4′-dimethyl-2′-propyl[2,6′-bi-1H-benzimidazol]-1′-yl)methyl]-[1,1′- biphenyl]-2-carbonitrile (compound VI) (5 g) and ethanol (40 ml). The reaction mixture was stirred at room temperature and 47% sodium hydroxide was added (2.34 ml) followed by water (20 ml), and the mixture was refluxed for 16 hours to afford a suspension. Then, the mixture was cooled to 25° C., stirred for 1 hour at 25° C. and then for 1 hour at 5° C. The thus formed solid was filtered off and washed with a cold mixture of 2/1 ethanol/water. The crystals were dried at 50° C. to afford 4.7 g of the desired product in 90% yield, having a purity of 98.9% (by HPLC).

### EXAMPLE 2

This example illustrates a process for preparing Telmisartan.

A two-necked reaction vessel equipped with a reflux condenser and a thermometer was charged with compound VII (10 g), potassium hydroxide powder (10 g), and propylene glycol (100 ml). The reaction mixture was refluxed at a temperature of about 150° C. overnight. The majority of the propylene glycol was distilled off under vacuum and the mixture was cooled to 85° C. Water was added in portions to afford a solution and charcoal was added (0.5 g). Stirring was maintained for 15 minutes at 85° C. and the hot mixture was filtered through a Celite pad. The filtrate was transferred into a clean reaction vessel and a solution of acetic acid (9.2 ml) in water (20 ml) was added in portions at 85° C. Stirring was maintained for 15 minutes at 85° C. and the suspension was cooled to 25° C. Stirring was maintained for one hour at 25° C. and a solid was obtained by filtration. The solid was washed with water followed by addition of water (100 ml) and the thus formed suspension was stirred for one hour at 85° C. and filtered. The cake was washed with hot water, dried at 80° C. in vacuum to afford 6.2 g of dry crude Telmisartan in 84% yield, having a purity of 99.3% (by HPLC), and containing less than 0.01% residual ammonia.

### EXAMPLE 3

This example demonstrates a process for crystallizing Telmisartan.

In a 500 ml three-necked round bottom flask equipped with a reflux condenser, a thermometer and a magnetic stirrer, crude Telmisartan (58.4 g) was suspended in DMF (293 ml). The suspension was heated to 90° C. using an oil bath, and left to cool down to 25° C. Mixing was maintained at this temperature for about an hour. Then, the mixture was cooled down to 5° C. and mixing was maintained at this temperature for about an hour. The solid was obtained by filtration, washed with cold ethanol and dried under vacuum to afford 47.9 g of the dried material in 82% yield, having a purity of 99.9% (by HPLC).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for preparing Telmisartan, the process comprising:
converting 4′-[(1,4′-dimethyl-2′-propyl[2,6′-bi-1H-benzirnidazol]-1′-yl)methyl]-[1,1′-biphenyl]-2-carboxamide (compound VII) into Telmisartan;
isolating the crude Telmisartan; and
optionally crystallizing the Telmisartan.

2. The process of claim 1, further comprising:
reacting 4′-[(1,4′-dimethyl-2′-propyl [2,6′-bi-1H-benzimidazol]-1′-yl)methyl]-[1,1′-biphenyl]-2-carbonitrile (compound VI) with an acid or a base in at least one solvent to produce 4′-[(1,4′-dimethyl-2′-propyl [2,6′-bi-1H-benzimidazol]-1′-yl)methyl]-[1,1′- biphenyl]-2-carboxamide (compound VII) as a solid;
isolating the solid product by filtration; and
optionally washing the solid with at least one solvent and drying the solid.

3. The process of claim 2, wherein the base is sodium hydroxide, potassium hydroxide, lithium hydroxide, barium hydroxide, cesium hydroxide, sodium bicarbonate, potassium bicarbonate, sodium carbonate, potassium carbonate, cesium carbonate, or a combination thereof.

4. The process of claim 3, wherein the base is sodium hydroxide.

5. The process of claim 2, wherein the acid is a mineral acid or an organic acid.

6. The process of claim 2, wherein the at least one solvent is methanol, ethanol, 1-propanol, 2-propanol, water or a mixture thereof.

7. The process of claim 6, wherein the at least one solvent is a mixture of ethanol and water.

8. The process of claim 2, wherein the solvent used for optionally washing the obtained solid is methanol, ethanol, 1-propanol, 2-propanol, water or a mixture thereof.

9. The process of claim 8, wherein the solvent is a 2:1 (v/v) mixture of ethanol/water.

10. The process of claim 2, wherein the compound VII is obtained as a crude product in at least about 90% yield and a purity of at least about 98.9%.

11. The process of claim 1, comprising:
reacting compound VII with a base in an organic solvent;
optionally distilling off a majority of the solvent and adding an anti-solvent;
optionally treating the reaction mixture with charcoal and Celite and filtering;
adding aqueous acetic acid to obtain a suspension;
cooling the suspension and isolating the Telmisartan as a solid product by filtration;
washing the Telmisartan with at least one solvent; and
drying the Telmisartan.

12. The process of claim 11, wherein the anti-solvent is water.

13. The process of claim 11, wherein the base is sodium hydroxide, potassium hydroxide, lithium hydroxide, barium hydroxide, cesium hydroxide, or a combination thereof.

14. The process of claim 13, wherein the base is potassium hydroxide.

15. The process of claim 11, wherein the reaction solvent is methanol, ethanol, 1-propanol, 2-propanol, N,N-dimethylacetamide (DMA), water, THF, ethylene glycol, propylene glycol, or a mixture thereof.

16. The process of claim 15, wherein the solvent is propylene glycol.

17. The process of claim 11, wherein the solvent used for washing the Telmisartan is methanol, ethanol, 1-propanol, 2-propanol, water or a mixture thereof.

18. The process of claim 17, wherein the solvent used for washing the Telmisartan is water.

19. The process of claim 11, wherein the crude Telmisartan is obtained in a yield of at least 84% and a purity of at least about 99.3%.

20. Telmisartan containing less than about 0.25% residual ammonia.

21. Telmisartan containing less than about 0.1% residual ammonia.

22. Telmisartan containing less than about 0.02% residual ammonia.

23. 4′-[(1,4′-dimethyl-2′-propyl [2,6′-bi-1H-benzimidazol]-1′-yl)methyl]-[1,1′-biphenyl]-2-carboxamide of the formula VII: [chemical structure]
